(19) United States
(12) Patent Application Publication
(10) Pub. No.: US 2006/0124128 A1
(43) Pub. Date: Jun. 15, 2006

(54) Portable intelligent controller for therapeutic gas systems

(76) Inventors: Geoffrey Frank Deane, Goleta, CA (US); Brenton Alan Taylor, Kenwood, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/274,755

(22) Filed: Nov. 14, 2005

Related U.S. Application Data
(60) Provisional application No. 60/627,735, filed on Nov. 12, 2004.

(51) Int. Cl. A61M 16/00; A62B 7/04; A62B 7/00

(52) U.S. Cl. 128/204.21; 128/204.26; 128/204.18

(57) ABSTRACT

A apparatus for delivering oxygen to a patient is provided. The apparatus includes a gas source such as an oxygen concentrator and a portable intelligent controller that is movable relative to the gas source and operable over a distance from the gas source. The portable intelligent controller is compact, lightweight and configured to remotely monitor and control one or more functions of the gas source. The functions include compressor speed, product gas production rate, valve timing, power supply and the like. The portable intelligent controller also has a user interface which allows the user to remotely adjust one or more settings of the gas source. In some implementations, the portable intelligent controller also includes a satellite conserver, which delivers oxygen in metered amounts in response to sensed breaths of the patient.

### CROSS-REFERENCE TO RELATED APPLICATIONS

The present application claims priority benefit under 35 U.S.C. § 119(e) from U.S. Provisional Application No. 60/627,735, filed Nov. 12, 2004, entitled SATELLITE CONSERVER FOR THERAPEUTIC GAS SYSTEMS, the entirety of which is hereby incorporated herein by reference.

### BACKGROUND OF THE INVENTION

1. Field of the Invention

This invention relates generally to therapeutic gas systems such as oxygen concentrators, more particularly, to a therapeutic gas system having a portable intelligent controller that can be used to remotely adjust one or more functions of the system.

2. Description of the Related Art

Various therapeutic gas systems have been developed to provide supplemental oxygen to patients who suffer from respiratory ailments such as Chronic Obstructive Pulmonary Diseases (COPD). Oxygen is often supplied to the patients by oxygen concentrators which produce oxygen concentrated air on a constant basis by filtering ambient air through a molecular sieve bed. A particularly useful class of oxygen concentrators is designed to be portable, allowing users to move about and to travel for extended period of time without the need to carry a supply of stored oxygen. Such portable concentrators are usually required to be small and light in order to be effective.

Oxygen concentrators in general are implicitly limited in terms of the rate at which they can deliver oxygen to the patient, but benefit because they are only duration-limited by their access to electric power. To make the portable concentrators small and light, the rate at which oxygen is concentrated by the device is further restricted. However, use of a device called a conserver mitigates this limitation as the conserver is designed to control and meter the delivery of oxygen to the patient.

The conserver, many designs of which are known in the art, senses a patient's breath demand and responds by delivering a volume of oxygen-rich gas (known as a bolus) to the patient. In most cases, the conserver is physically part of or directly attached to the oxygen source such as an oxygen tank or concentrator. Therefore, in order to achieve reliable breath detection and bolus delivery, the hose between the oxygen source and the patient is usually relatively short. The length of the hose is limited to ensure that the pressure drops in the hose do not reduce breathing pressure signals, thereby degrading breath detection.

Applicant's co-pending U.S. patent application Ser. No. 10/962,194 discloses a satellite conserver system developed to address the shortcomings of conventional conservers. The satellite conserver system is preferably a small and compact unit, about the size of a personal digital assistant (PDA), and thus can be easily carried by the patient. The satellite conserver allows the breathing sensing and gas metering functions to be performed remotely from the base unit. As such, patients may use a much longer hose to connect to the oxygen source, which greatly increases patient convenience. Applicant's co-pending U.S. patent application Ser. No. 11/170,743 discloses a satellite conserver system configured with intelligent bolus volume and timing control to provide the users with additional benefit regardless of the oxygen source.

It is further desirable to provide patients the ability to remotely adjust and control various functions of the oxygen source. To this end, there is a particularly need to provide a compact, portable intelligent controller that can be used to remotely adjust or control one or more functions of the therapeutic gas systems.

### SUMMARY OF THE INVENTION

In one aspect, the preferred embodiments of the present invention provide an apparatus for delivering oxygen to a patient. The apparatus comprises an oxygen concentrator having an oxygen delivery outlet, a flexible tube having a length, one end of said tube connected to receive oxygen from said outlet, a conserver which delivers oxygen in metered amounts in response to sensed breaths of the patient, said conserver being connected to (i) receive oxygen from the other end of the tube and (ii) deliver the oxygen to the patient; and a controller which controls one or more functions of the concentrator, the controller being movable relative to the oxygen source and operable over a distance from the oxygen source, said distance is substantially equal to or greater than the length of the flexible tube. The functions controlled by the controller are preferably selected from the group consisting of compressor speed, valve timing, flow rate, gas production rate, supply voltage or current, and combinations thereof. In one embodiment, the controller further comprises a user interface wherein the user interface is configured for the patient to remotely adjust one or more settings of the oxygen concentrator. In another embodiment, the controller also controls one or more functions of the conserver, which includes controlling the timing of one or more conserver valves. In yet another embodiment, the controller communicates with the oxygen concentrator by a communication link selected from the group consisting of electronic cable, wireless electronic communication, infrared communication, radio control communication, and combinations thereof. Preferably, the communication link between the controller and the concentrator is external to the concentrator. In another embodiment, the controller is in communication with external respiratory care diagnostic tools, preferably selected from the group consisting of oximeters, spirometers, and combinations thereof. In yet another embodiment, the flexible tube has a length of greater than 10 feet.

In another aspect, the preferred embodiments of the present invention provide a method of producing a therapeutic gas. The method comprises providing an oxygen concentrator having a plurality of settings which control the function of the concentrators and adjusting the function of the concentrator by generating a signal at a distance from the concentrator wherein the signal is generated by a programmable controller, propagating the signal over the distance, using the concentrator to sense the signal, and altering one or more of the settings in response to sensing of the signal by the concentrator. In one embodiment, adjusting the function of the concentrator comprises adjusting a concentrator operating parameter selected from the group consisting of compressor speed, valve timing, flow rate, gas production rate, supply voltage or current, and combinations thereof. In another embodiment, propagating the signal comprises propagating an electric signal using a method selected from the group consisting of electronic cable interface, wireless communication, and combinations thereof.

In yet another aspect, the preferred embodiments of the present invention provide an apparatus for delivering therapeutic gas to a patient. The apparatus comprises a therapeutic gas source, a portable intelligent controller, a communication interface between the gas source and the controller. Preferably the controller monitors and controls one or more functions of the therapeutic gas source by communicating with the gas source via the communication interface. In one embodiment, the controller weights less than 5 lbs and has a length of less than or equal to 5.25 inches, a width of less than or equal to 3.25 inches. Preferably, the controller is operable over a distance from the gas source wherein the distance is substantially equal to or greater than about 10 feet. In one embodiment, the portable intelligent controller comprises a satellite conserver. In another embodiment, the communication interface between the gas source and the controller is selected from the group consisting of oxygen concentrators, oxygen gas cylinders, and liquid oxygen reservoirs.

In yet another aspect, the preferred embodiments of the present invention provide a satellite conserver, in communication with a gas source, for a therapeutic gas delivery system. The conserver comprises a breath sensor, a gas control valve, a programmable controller having a user interface. Preferably, the satellite conserver is movable relative to the gas source and operable over a distance from the gas source, wherein the program controller communicates information with the gas source, monitors and controls one or more process parameters of the gas delivery system, wherein the user interface allows users to adjust one or more of the parameters of the gas delivery system. In one embodiment, the conserver further comprises a power source. In another embodiment, the conserver communicates information to the gas source to change oxygen production in response to oxygen delivery to the patient. In yet another embodiment, the information communicated between the programmable controller and the gas source is selected from the group consisting of compressor speed, valve timing, supply voltage or current, concentrator power consumption, concentrator battery levels, oxygen concentration, conserver power usage, conserver battery levels, patient breathing rates, patient selectable flow rate, and combinations thereof. Preferably, the information is communicated by a system selected from the group consisting of electronic interface by cable, infrared, pneumatic, wireless radio, and combinations thereof. In some implementations, the gas source comprises a base unit concentrator wherein the base unit concentrator provides at least one of gas supply, error reporting for gas production processes, and limited external communication. In other implementations, the programmable controller communicates information with the gas source via an electronic communication interface, wherein the information is selected from the group consisting of conserver power, valve sensor settings, patient interface settings, and gas source control parameters. In yet another implementation, the programmable controller communicates information with the gas source via a pneumatic interface. In certain embodiments, the conserver provides a function selected from the group consisting of bolus delivery to the patient, patient interface, error reporting, external data communication, data logging, and combinations thereof. In certain preferred embodiment, the conserver further comprises a second communication interface, wherein the second communication interface is configured to establish communication between the conserver and external diagnostic devices.

### BRIEF DESCRIPTION OF THE DRAWINGS

FIG. 1 is a schematic illustration of a therapeutic gas system of one preferred embodiment of the present invention;

FIG. 2 is a schematic illustration of a therapeutic gas system of another preferred embodiment of the present invention which incorporates an oxygen concentrator as the gas source and a satellite conserver as part of the portable intelligent controller;

FIG. 3 is a schematic illustration of the system of FIG. 2, showing the details of the satellite conserver;

FIG. 4 is a schematic illustration of the therapeutic gas system of another preferred embodiment, showing the interface between the base unit and the portable intelligent controller;

FIG. 5 is a schematic illustration of the therapeutic gas system of another preferred embodiment, showing the interface between the portable intelligent controller and external respiratory care diagnostic tools; and

FIGS. 6A and 6B illustrate the manner in which the portable intelligent controller can be worn around a belt clip and worn on a neck or shoulder strap.

### DETAILED DESCRIPTION OF THE PREFERRED EMBODIMENT

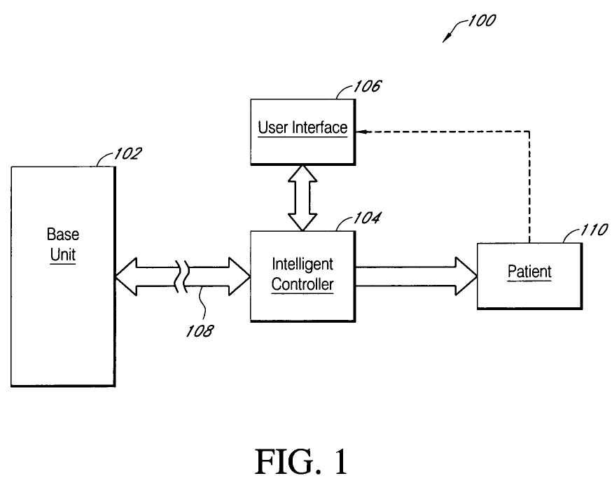

FIG. 1 schematically illustrates a therapeutic gas system 100 of one preferred embodiment of the present invention. As shown in FIG. 1, the system 100 generally includes a base unit 102, a portable intelligent controller 104, a user interface 106 connected to the controller, and a communication link 108 between the portable intelligent controller 104 and the base unit 102 which provides transmission of information and/or commands between the controller and the base unit. The base unit 102 is a therapeutic gas source, preferably one that provides a therapeutic gas with a high oxygen concentration. The base unit 102 can comprise a variety of different devices including but not limited to a stationary or portable oxygen concentrator, oxygen gas cylinder, liquid oxygen reservoirs or the like. As will be described in greater detail below, one or more functions of the base unit 102 can be remotely adjusted and controlled by the patient via the portable intelligent controller 104.

The portable intelligent controller 104 is preferably compact, lightweight and movable relative to the base unit 102. In one embodiment, the dimension and weight of the intelligent controller 104 are similar to those of a cellular phone or personal digital assistant (PDA) so that the controller 104 can be easily and conveniently carried by the patient 110. In one implementation, the portable intelligent controller 104 weighs less than 5 lbs, preferably less than 3 lbs, more preferably less than 2 lbs. In another implementation, the portable intelligent controller 104 has a length of less than or equal to 4 inches, a width of less than or equal to 4 inches, and a thickness of less than or equal to 1 inch. In certain preferred embodiments, the portable intelligent controller 104 functions as the brain of the therapeutic gas system by performing a variety of different functions such as controlling the delivery and metering of the gas flow to the patient, adjusting rate of gas production based on process conditions, monitoring and recording various parameters of the system, and allowing the patient to adjust various settings through the user interface 106 attached thereto.

The portable intelligent controller 104 communicates with the base unit through the communication link 108. The communication link 108 can be based on a variety of different systems and technologies including but not limited to electronic interface by cable, infrared systems, pneumatic systems, wireless radio, voice recognitions, or other technologies. Preferably, the communication link 108 is located external to the base unit 102, which allows the portable intelligent controller 104 to operate remotely at a distant from the base unit 102.

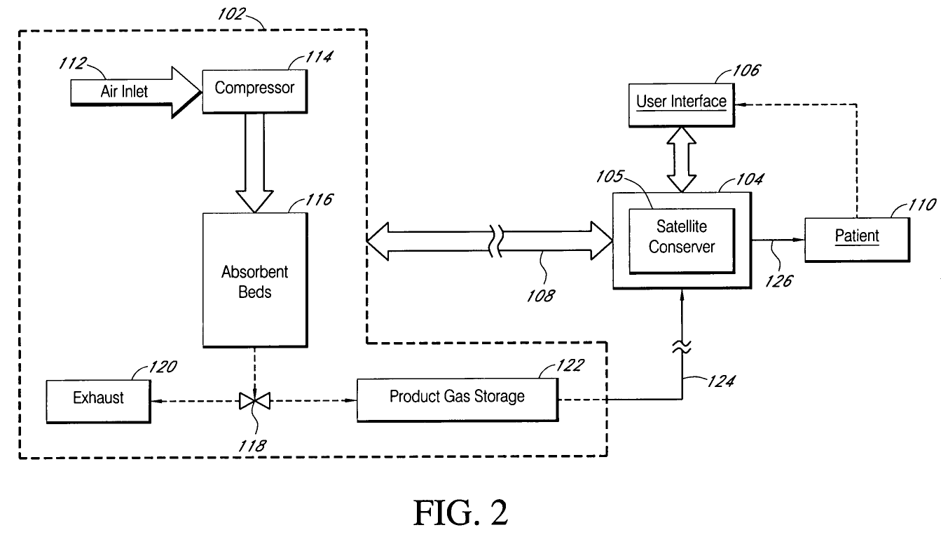

FIG. 2 schematically illustrates a preferred embodiment of the system 100 in which the base unit 102 comprises an oxygen concentrator and the portable intelligent controller 104 comprises a satellite conserver. As shown in FIG. 2, the oxygen concentrator 102 generally includes an air inlet 112, a compressor 114, a plurality of adsorbent beds 116, valves 118, an exhaust port 120, and product gas storage 122. In a preferred embodiment, the programmable controller for the oxygen concentrator is not included in the base unit 102 so that the functions of the concentrator can be adjusted remotely by the patient.

The general function and operation of oxygen concentrators are known in the art and therefore will only be briefly discussed below. In the oxygen concentrator, the air inlet 112 provides air to the compressor 114 through various filters. The compressed air is routed through the adsorbent beds 116 in accordance with a pressure swing adsorption (PSA) cycle, which typically selectively adsorbs one or more atmospheric components in the compressed air, leaving a product gas with a higher concentration of the remaining, un-adsorbed components. A portion of the product gas is subsequently routed to fill the product storage 122 while another portion is used to recharge the adsorbent material in the adsorbent beds 116. The waste gas, typically nitrogen rich, is exhausted from the system through the exhaust port 120. The arrangement of adsorbent beds, valving, PSA cycles can vary based on the concentrator design. Process variables such as valve timing, gas flow rates, and compressor speed are often adjusted to optimize the production of gas based on the patient's need and other process conditions. Further details of the workings of concentrator based oxygen therapy systems are described in Applicant's co-pending U.S. Pat. No. 10/962,194, which is incorporated by reference in its entirety.

As FIG. 2 further illustrates, the portable intelligent controller 104 comprises a satellite conserver 105. The satellite conserver 105 is configured to deliver oxygen in metered amounts to the patient 110 in response to sensed breaths of the patient. As shown in FIG. 2, the satellite conserver 105 is connected to the product gas storage 122 of the oxygen concentrator 102 by a gas conduit 124 such as a flexible tube. The length of the gas conduit 124 is preferably greater than 10 feet, preferably greater than 20 feet, preferably greater than 50 feet. The satellite conserver 104 is also configured to deliver bolus of oxygen to the patient 110 via a flexible tube 126.

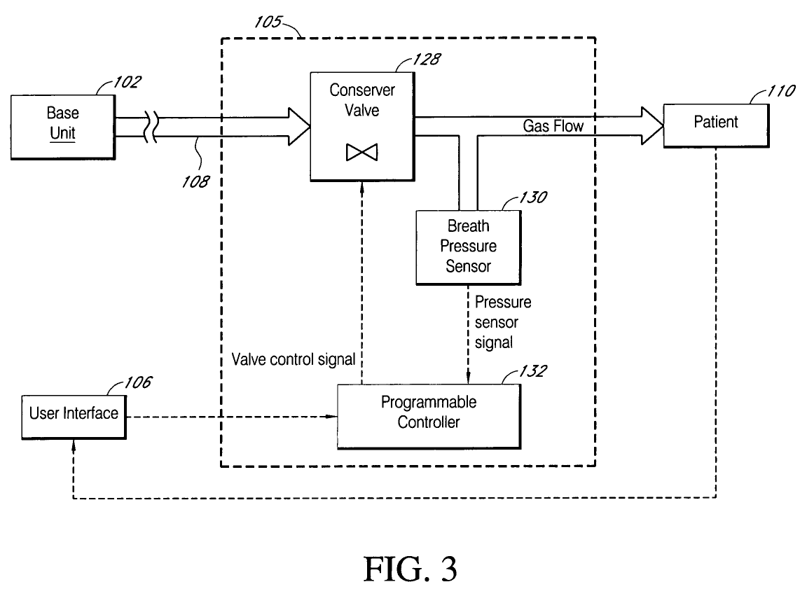

As shown in FIG. 3, the satellite conserver 105 generally includes a valve 128, a breath pressure sensor 130, and a programmable controller 132 that is connected to the user interface 106. Oxygen rich air is supplied from the base unit 102 through the valve 128. The breath pressure sensor 130 detects the presence of a breath which causes the valve 128 to deliver a bolus of oxygen rich air to the patient 110. In one embodiment, considerable intelligence is employed to optimize the relation between the breath pressure sensor 130 input and the timing of the valve 128 and the bolus volume delivered to patient 110, as described in Applicant's co-pending U.S. patent application Ser. No. 11/170,743, which is incorporated by reference in its entirety.

In a preferred implementation, the programmable controller 132 in the satellite conserver 105 provides this intelligent control at the point of application to the patient as it is often desirable to change the oxygen concentrator's oxygen production rate in response to the rate at which oxygen is being delivered to the patient. In some embodiments, changing the oxygen concentrator's production rate may require changing the speed of the compressor or changing other operating parameters of the base unit 102 such as valve timing, voltage or current supply to components, or net power consumption. Thus, in a preferred implementation, the programmable controller 132 of the satellite conserver 105 communicates information to and from the base unit via the communication link 108. This communication may be electronic, pneumatic, infrared, radio transmission, satellite link, cellular telephony, or by a combination of these methods. The portable intelligent controller 104, which comprises the satellite conserver 105, provides a level of patient control of the oxygen concentrator 102 functionality while the patient is at a distance from the concentrator. As such, it is advantageous to allow the patient to change settings on the concentrator without the necessity of returning to the oxygen source. It will be further understood that, in some embodiments, the programmable controller designed to remotely communicate with and control the oxygen concentrator is independent from the programmable controller of the satellite conserver. Moreover, in certain implementations, the portable intelligent controller and the satellite conserver are two separate components housed in different enclosures.

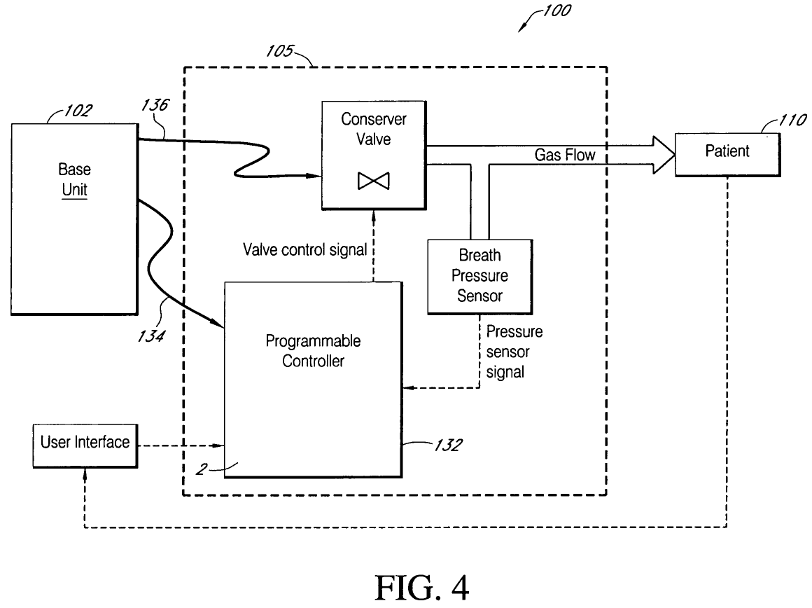

FIG. 4 illustrates another embodiment of the therapeutic gas system 100 in which the programmable controller 132 is configured to allow the user to adjust one or more functions of the base unit 102, which in this embodiment is an oxygen concentrator. As shown in FIG. 3, an electronic cable 134 extends between the satellite conserver 105 and the oxygen concentrator 102. The electronic cable 134 can be attached to an air tube 136 extending between the satellite conserver 105 and the oxygen concentrator 102. In this embodiment, the concentrator is the base unit 102 containing primarily the heavier components such as the compressor, adsorbent beds, product gas storage, while the satellite conserver 105 serves as a portable, compact oxygen delivery, diagnostic and main user interface unit.

In additional to gas flow between the base unit 102 and the satellite conserver 105, other communications between the two units include power to the conserver for the breath pressure sensors, valve timing, patient interface, patient interface settings, and valve/sensor status and control using known electronic, infrared, or other communication methods. In addition to bolus delivery through the cannula, the satellite conserver 105 can also interface with the patient 110 in an information sense, such as providing system error reporting, system diagnosis. In one embodiment, the base unit 102 provides oxygen, error reporting for internal functions, and limited external communication. The base unit 102 in some implementations has a transportable power source, such as a battery or a fuel cell. In one embodiment, the patient can remotely adjust the valve timing, compressor speed, flow rates and other settings of the base unit 102 through the user interface 106 of the satellite conserver 105.

Additionally, the programmable controller 132 is preferably capable of controlling complex breath detection and delivery scenarios and can assume many of the control functions typically resident in single unit oxygen sources. In one embodiment, the portable intelligent controller incorporating the satellite conserver 105 handles substantially the complete user interface, error reporting, data logging and reporting, and external communications. However, the portable intelligent controller still maintains a compact dimension, preferably less than 3.25 inches×5.25 inches×1 inch. In other preferred embodiments, the portable intelligent controller, including the satellite conserver, has substantially the same size as that of a cellular phone or a PDA. The portable intelligent controller, including the satellite conserver, may require appropriate sized power sources on the scale of a cell phone battery. The portable intelligent controller may be easily carried around, worn on a belt clip if desired, thereby permitting the patient to be essentially free of the base unit 102 most of the time.

In yet another embodiment, the operating information can be communicated pneumatically between the base unit 102 and the intelligent portable controller 104. Pressure and/or flow sensors on the satellite conserver 105 and the concentrator 102 can be monitored and variations in signal may be correlated to known conditions. For example, the concentrator may observe pressure drops in the gas conduit when a bolus is delivered. Based on the size and/or frequency of the pressure drops, it may determine breathing rates and flow settings, and adjust its product rate as needed. In this embodiment, no interface other than an air conduit is required.

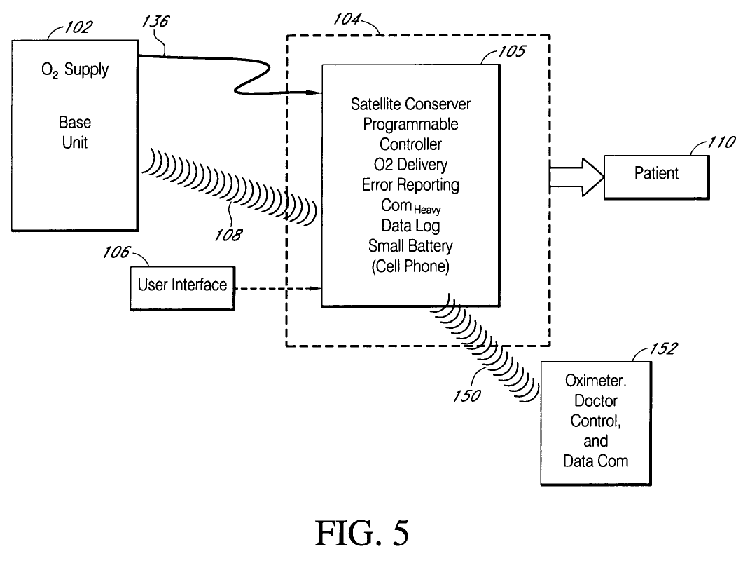

FIG. 5 illustrates another embodiment in which the portable intelligent controller 104 and the base unit concentrator 102 may communicate using a remote communication method 108 such as radio transmission (RF) or infrared transmission. As illustrated, the intelligent controller 104 in certain preferred embodiments includes the satellite conserver 105. In one implementation, the satellite conserver 105 and oxygen concentrator 102 may share information regarding flow settings, bolus delivery rates, pressures (internal and ambient), and other operating parameters which may enhance performance. It may also be desirable to have the satellite conserver 105 serve as the master in the communication protocol, wherein the satellite conserver 105 initiates and controls the communications. Alternatively, the base unit concentrator 102 may serve as the communication master.

In a preferred implementation, the base unit 102 and the satellite conserver 105 are able to operate in communicative isolation from each other when no information is communicated or the devices are unable to determine information from pneumatic signals. In this implementation, the satellite conserver 105 continues to deliver oxygen per the user adjustable flow setting, and that the base unit concentrator 102 assumes that the satellite conserver 105 is set to its maximum flow settings. This may be used to assure that oxygen delivery to the patient 110 does not exceed oxygen production by the concentrator.

In another embodiment, the oxygen concentrator 102 is reduced to a device that produces oxygen and the portable intelligent controller 104 is the primary means of controlling the delivery of the oxygen. In this implementation, the concentrator 102 is always used in conjunction with the satellite conserver 105 that is part of the portable intelligent controller 104, wherein oxygen flows from the concentrator to the satellite conserver and then is delivered in doses to the patient. In a further refinement of this embodiment, it may be possible for the intelligent controller 104 and/or the satellite conserver 105 to mechanically connect or dock to the base unit concentrator 102. While in this mode, the oxygen carrying tube connecting the two devices may be relatively short. In addition, while in this mode, it may be desirable that the two devices are in communication. When the satellite conserver 105 is docked into the base unit concentrator 102, a hard electronic connection may be established such that information may be communicated between the two devices. This may enable other communications methods, such as radio transmission, to be turned off, which is particularly useful for operation in radio-sensitive settings such as commercial aircraft. Power from the base unit concentrator 102 may also be used to operate or recharge the batteries on the satellite conserver. When the satellite conserver is removed from its docking position, a longer oxygen carrying tube may be used between the two devices, and the devices may employ one of the communications methods described above.

As FIG. 5 further shows, a second communication path 150, preferably wireless, can be used to gather information from other diagnostic devices 152, such as oximeters, spirometers, or other respiratory care diagnostic tools. This second path 150 can also provide communication to remote patient monitoring devices or care providers, and may also be used as a path for care providers to have remote control capability. This second path 150 may also be used to interface with other equipment such as ventilators or continuous positive airway pressure (CPAP) machines for sleep apnea.

In yet another embodiment, the satellite conserver 105 is equipped with data storage capability and acts as the communications hub for a system of inter-communicating devices. Devices 152 such as oximeters or electronic spirometers may be used periodically, and data generated by the devices may be stored in the satellite conserver. This data may be used by itself or in concert with other system data to adjust operating parameters of the satellite conserver, the concentrator, or other device in communication with the satellite conserver, or this data may be available for download and viewing by a healthcare professional.

Alternatively, the satellite conserver may serve as the communications hub, but may transfer said data to a second device in the communication network for storage. Alternatively, the base unit concentrator 102 may serve as the communications hub for the system of connected devices. In one version, the concentrator may store operating data from its own systems and from other devices in the communications network. In another version, this data may be relayed to one of the connected devices for external storage.

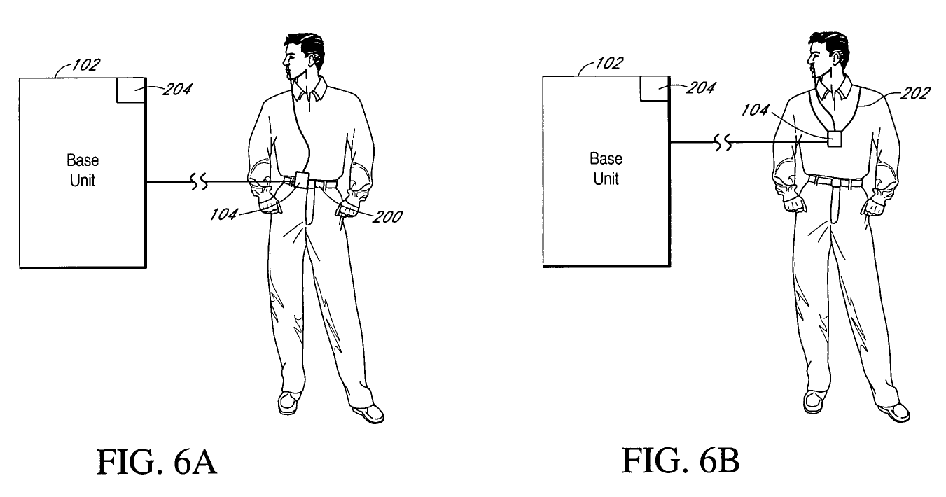

The portable intelligent controller is lightweight and can be easily carried by the patient. FIG. 6A illustrates the manner in which the lightweight, portable intelligent controller 104 can be conveniently worn on a belt clip 200. FIG. 6B illustrates the manner in which the portable intelligent controller 104 can be worn around the patient's neck via a strap 202. As FIGS. 6A and 6B further show, the base unit 102 preferably has a docking station 204 configured to receive the portable intelligent controller 104 which in some implementations includes the satellite conserver 105.

Although the foregoing description of certain preferred embodiments of the present invention has shown, described and pointed out the fundamental novel features of the invention, it will be understood that various omissions, substitutions, and changes in the form of the detail of the system, apparatus, and methods as illustrated as well as the uses thereof, may be made by those skilled in the art, without departing from spirit of the invention. Consequently, the scope of the present invention should not be limited to the foregoing discussions.

What is claimed is:

1. An apparatus for delivering oxygen to a patient, comprising:
an oxygen concentrator having an oxygen delivery outlet;
a flexible tube having a length, one end of said tube connected to receive oxygen from said outlet;
a conserver which delivers oxygen in metered amounts in response to sensed breaths of the patient, said conserver being connected to (i) receive oxygen from the other end of the tube and (ii) deliver the oxygen to the patient; and
a controller which controls one or more functions of the oxygen concentrator, the controller being movable relative to the oxygen concentrator and operable over a distance from the oxygen concentrator, said distance is substantially equal to or greater than the length of the flexible tube.

2. The apparatus of claim 1, wherein the controller comprises a user interface, said user interface is configured for the patient to remotely adjust one or more settings of the oxygen concentrator.

3. The apparatus of claim 1, wherein the controller controls one or more functions of the conserver.

4. The apparatus of claim 3, wherein the controller controls the timing of one or more conserver valves.

5. The apparatus of claim 1, wherein the controller controls one or more functions of the oxygen concentrator, said functions are selected from the group consisting of compressor speed, valve timing, flow rate, gas production rate, supply voltage or current, and combinations thereof.

6. The apparatus of claim 1, wherein the controller communicates with the oxygen concentrator by a communication interface selected from the group consisting of electronic cable, wireless electronic communication, infrared communication, radio control communication and combinations thereof.

7. The apparatus of claim 6, wherein said communication interface between the controller and the concentrator is external to the concentrator.

8. The apparatus of claim 1, wherein the controller is in communication with external respiratory care diagnostic tools.

9. The apparatus of claim 8, wherein the respiratory care diagnostic tools are selected from the group consisting of oximeters, spirometers, and combinations thereof.

10. The apparatus of claim 1, wherein the flexible tube has a length of at least 10 feet.

11. A method of producing a therapeutic gas, comprising:
providing an oxygen concentrator having a plurality of settings which control the function of said concentrator; and
adjusting the function of the concentrator by generating a signal at a distance from the concentrator wherein said signal being generated by a programmable controller, propagating the signal over said distance, using the concentrator to sense said signal, and altering one or more of said settings in response to sensing of said signal by the concentrator.

12. The method of claim 11, wherein adjusting the function of the concentrator comprises adjusting a concentrator operating parameter selected from the group consisting of compressor speed, valve timing, flow rate, gas production rate, supply voltage or current, and combinations thereof.

13. The method of claim 12, wherein propagating said signal comprises propagating an electric signal using a method selected from the group consisting of electronic cable interface, wireless communication, and combinations thereof.

14. An apparatus for delivering therapeutic gas to a patient, comprising:
a therapeutic gas source having a therapeutic gas delivery outlet;
a portable intelligent controller;
a communication interface between said gas source and controller;
wherein the controller monitors and controls one or more functions of the therapeutic gas source by communicating with the gas source via the communication interface, said controller weighing less than 5 lbs and having a length of less than or equal to 5.25 inches and a width of less than or equal to 3.25 inches, said controller is operable over a distance from the gas source, said distance being substantially equal to or greater than about 10 feet.

15. The apparatus of claim 14, wherein said portable intelligent controller comprises a satellite conserver.

16. The apparatus of claim 14, wherein said communication interface is selected from the group consisting of electronic cable, infrared, wireless radio, and combinations thereof.

17. The apparatus of claim 14, wherein said portable intelligent controller further comprising a user interface, said user interface allows the patient to remotely adjust one or more functions of the gas source.

18. The apparatus of claim 15, wherein said gas source is selected from the group consisting of oxygen concentrators, oxygen gas cylinders, and liquid oxygen reservoirs.

19. A satellite conserver, in communication with a gas source, for a therapeutic gas delivery system, comprising:
a breath sensor;
a gas control valve;
a programmable controller having a user interface; and
wherein the satellite conserver is movable relative to the gas source and operable over a distance from the gas source, wherein the program controller communicates information with the gas source, monitors and controls one or more process parameters of the gas delivery system, wherein said user interface allows users to adjust one or more of said parameters of the gas delivery system.

20. The conserver of claim 19, further comprising a power source.

21. The conserver of claim 19, wherein the gas source comprises a base unit concentrator.

22. The conserver of claim 19, wherein the conserver communicates information to the gas source to change oxygen production in response to oxygen delivery to the patient.

23. The conserver of claim 22, wherein the information is selected from the group consisting of compressor speed, valve timing, supply voltage or current, concentrator power consumption, concentrator battery levels, oxygen concentration, conserver power usage, conserver battery levels, patient breathing rates, patient selectable flow rate, and combinations thereof.

24. The conserver of claim 19, wherein the information is communicated by a system selected from the group consisting of electronic interface by cable, infrared, pneumatic, wireless radio, and combinations thereof.

25. The conserver of claim 19, wherein the base unit concentrator provides at least one of: gas supply, error reporting for gas production processes, and limited external communication.

26. The conserver of claim 19, wherein the programmable controller communicates information with the gas source via an electronic communication interface, said information is selected from the group consisting of conserver power, valve sensor settings, patient interface settings, and gas source control parameters.

27. The conserver of claim 19, wherein the programmable controller communicates information with the gas source via a pneumatic interface.

28. The conserver of claim 19, wherein the conserver provides a function selected from the group consisting of bolus delivery to the patient, patient interface, error reporting, external data communication, data logging, and combinations thereof.

29. The conserver of claim 19, wherein the conserver can be worn on a belt clip.

30. The conserver of claim 19, further comprising a second communication interface, said second communication interface is configured to establish communication between said conserver and external diagnostic devices.
